(19) United States
(12) Patent Application Publication
(10) Pub. No.: US 2019/0275179 A1
(43) Pub. Date: Sep. 12, 2019

(54) SONOPHORE-LABELED PROBES AND RELATED IN VIVO IMAGING TECHNIQUES

(71) Applicant: MEMORIAL SLOAN KETTERING CANCER CENTER, New York, NY (US)

(72) Inventors: Jan GRIMM, New York, NY (US); Thomas REINER, New York, NY (US); Christian BRAND, New York, NY (US); Katja HAEDICKE, New York, NY (US)

(73) Assignee: MEMORIAL SLOAN KETTERING CANCER CENTER, New York, NY (US)

(21) Appl. No.: 16/347,898

(22) PCT Filed: Nov. 10, 2017
(86) PCT No.: PCT/US2017/061192

§ 371 Date: May 7, 2019

Related U.S. Application Data
(60) Provisional application No. 62/421,123, filed on Nov. 11, 2016.

(51) Int. Cl. A61K 49/22

(52) U.S. Cl. CPC A61K 49/221; A61K 49/227

(57) ABSTRACT

The present technology provides compositions and nanoemulsions useful in optoacoustic imaging. A composition of the present technology includes at least one contrast agent covalently linked to a targeting agent; where the at least one contrast agent includes a dark quencher; the targeting agent includes an antibody, an antigen, an antigen-targeting ligand, a receptor ligand, or an adhesion peptide; and the at least one contrast agent and targeting agent are covalently linked by a linker of formula (I) X¹-L¹-X² (I) where X1 is an amino, amide, S, or O of the at least one contrast agent; L¹ is C₁-C₁₂ alkylene, C₂-C₁₂ alkenylene, C₅-C₁₂ cycloalkylene, or C₆-C₁₂ arylene; and X² is an amino, amide, S, or O of the targeting agent.

### CROSS-REFERENCE TO RELATED APPLICATION

This application claims priority to U.S. Provisional Application No. 62/421,123, filed Nov. 11, 2016, the contents of which are incorporated herein by reference in their entirety.

### U.S. GOVERNMENT RIGHTS

This invention was made with government support under EB014944 and CA008748 awarded by the National Institutes of Health. The government has certain rights in the invention.

### FIELD

The present technology relates generally to compositions and methods suitable for obtaining optoacoustic imaging. More particularly, and not by way of limitation, the present technology relates to compositions and methods suitable for obtaining tumor specific optoacoustic images in vivo.

### BACKGROUND

Optoacoustic imaging is a growing field. Optoacoustic imaging systems image the optical absorption of fluorochromes and biomolecules deep in biological tissues using the generated ultrasound waves. The possibility of using non-invasive modalities (e.g., optoacoustic imaging) allows for use of contrast agents for better insight into tumor physiology. However, the availability of tumor specific exogenous contrast agents is still limited. While near-infrared fluorescent dye-labeled probes have been used for optical imaging, these dyes mainly emit absorbed energy in the form of fluorescence, making only a partial amount of energy available for a strong optoacoustic signal. Thus, a need exists for contrast agents capable of generating strong optoacoustic signals and having tumor specificity.

### SUMMARY

In an aspect, a composition is provided that includes at least one contrast agent covalently linked to a targeting agent; where the at least one contrast agent includes a dark quencher; the targeting agent includes an antibody, an antigen, an antigen-targeting ligand, a receptor ligand, or an adhesion peptide; and the at least one contrast agent and targeting agent are covalently linked by a linker of formula (I)

X¹-L¹-X² (I)

where X¹ is a N, S, or O of the at least one contrast agent; L¹ is C₁-C₁₂ alkylene, C₂-C₁₂ alkenylene, C₅-C₁₂ cycloalkylene, or C₆-C₁₂ arylene; and X² is a N, S, or O of the targeting agent.

In an aspect, the present technology provides an oil-in-water nanoemulsion composition that includes a plurality of lipid micelles in an aqueous phase. Each lipid micelle includes an interior and an exterior of the micelle; an outer amphiphilic component in contact with the aqueous phase on the exterior and with an oil phase on the interior; and at least one contrast agent within the interior. The plurality of lipid micelles exhibit an intensity-weighted average particle diameter of about 100 nm to about 500 nm as determined by dynamic light scattering.

### BRIEF DESCRIPTION OF THE DRAWINGS

FIGS. 1A-1D illustrate the synthesis of BHQ-1-cRGD and spectroscopic characterization for BHQ-1-cRGD. FIG. 1A illustrates the synthetic schematic of BHQ-1-cRGD. FIG. 1B illustrates the mass spectrometry analysis (positive polarized) of BHQ-1-cRGD. FIG. 1C illustrates the HPLC chromatogram for BHQ-1-cRGD and absorbance peaks for RGD (280 nm) and BHQ-1 (534 nm), respectively. FIG. 1D illustrates the absorption spectrum of BHQ-1 (solid line) and BHQ-1-cRGD (dotted line).

FIG. 2A illustrates a white light image of a 0-500 μM dilution series of BHQ-1 in agarose phantoms. FIG. 2B illustrates RSOM image of the agarose phantoms with the 0-500 μM dilution series of BHQ-1. FIG. 2C illustrates a graph of the detected optoacoustic signal in BHQ-1 agarose phantoms as a function of BHQ-1 concentration.

FIGS. 3A and 3B illustrate the in vitro optoacoustic imaging of U-87 spheroid cells. FIG. 3A illustrates RSOM images of U-87 spheroids after 2 h incubation without (native), with 50 μM BHQ-1-cRGD, and with M BHQ-1-cRGD and 100-fold excess free cRGD (block). FIG. 3B illustrates a graph of the detected optoacoustic signal in U-87 spheroids.

FIGS. 4A and 4B illustrate the in vivo optoacoustic imaging of mouse footpads. FIG. 4A illustrates the RSOM images of mouse footpad before and after subcutaneous injection of BHQ-1 (red=low frequencies; green=high frequencies; yellow=overlay). FIG. 4B illustrates a graph of detected optoacoustic signal at the injection site compared to the footpad background.

FIGS. 5A and 5B illustrate the ex vivo optoacoustic imaging of tumors after intravenous injection of BHQ-1-cRGD and sodium chloride. FIG. 5A illustrates RSOM images of U-87 tumors ex vivo 2 h after intravenous injection of 50 nmol BHQ-1-cRGD or sodium chloride, respectively (red=low frequencies; green=high frequencies; yellow=overlay). FIG. 5B illustrates a profile plot through the U-87 tumors after BHQ-1-cRGD (top) or sodium chloride (bottom).

FIGS. 6A-6D illustrate preparation and characterization of nanoemulsions according to the present technology. FIG. 6A illustrates the preparation of nanoemulsions as provided in Example 4. FIG. 6B illustrates the structure of nanoemulsion containing lipids, oil and contrast agents. FIG. 6C illustrates the photophysical characterizations of nanoemulsions showed variation between the UV/Vis and optoacoustic spectra. FIG. 6D illustrates the variation in the shape of the UV/Vis and optoacoustic spectra for NE-IRDye QC1, NE-IR780, and NE-ICG, respectively.

FIGS. 7A and 7B illustrate the optoacoustic library and characterization of nanoemulsions in tissue mimicking phantoms. FIG. 7A illustrates the nanoemulsion compositions according to the present technology encapsulating exemplary contrast agents. FIG. 7B illustrates MSOT imaging of phantoms for serial diluted nanoemulsions, with ICG (3 μM) included as a reference standard, with the optoacoustic intensity is shown at the maximum peak absorbance wavelength for each nanoemulsion.

FIGS. 8A-8C illustrate the ex vivo optoacoustic imaging of NE-IRDye QC1 and NE-IR780 in a 4T1 tumor model, 24 h after intravenous injections using the multi-spectral optoacoustic tomography (MSOT). FIG. 8A illustrates the contrast agent and nanoemulsion concentration of NE-IRDye QC1 and NE-IR780 used for ex vivo imaging of Example 5. FIG. 8B illustrates ex vivo optoacoustic image reconstruction of NE-IRDye QC1 (left) and quantifications (right) between non-injected and injected mice. FIG. 8C illustrates ex vivo optoacoustic image reconstruction of NE-IR780 (left) and quantifications (right) between the non-injected and injected mice.

FIGS. 9A-9C illustrate in-vivo accumulation of nanoemulsion IRDye QC-1 in a 4T1 tumor model 24 h after intravenous injection using multi-spectral optoacoustic tomography (MSOT). FIG. 9A illustrates the transverse MSOT image of a 4T1 tumor-bearing mouse (n=3) at time points t=0 h and t=24 h after injection of NE IRDye QC-1 and overlaid with NE-IRDye QC-1 signature scoring using a direct least-squares (DCLS) model based technique. FIG. 9B illustrates the overall optoacoustic intensity spectra from 680-900 nm at the tumor region before and after NE-IRDye QC1 injection (left) compared to NE-IR780 (right). FIG. 9C illustrates the average optoacoustic signal intensities before and after intravenous injections for NE-IRDye QC1 (left) and NE-IR780 (right).

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups may be substituted or unsubstituted. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. Heterocyclyl groups may be substituted or unsubstituted. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. The phrase includes heterocyclyl groups that have other groups, such as alkyl, oxo or halo groups, bonded to one of the ring members, referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups may be substituted or unsubstituted. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, N.Y., (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O⁻. A “hydroxyalkyl” group is a hydroxyl-substituted alkyl group, such as HO—CH₂—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “isocyano” refers to —NC.

The term “isothiocyano” refers to —NCS.

The term “pentafluorosulfanyl” refers to —SF₅.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g. alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g. Na⁺, Li⁺, K⁺, Ca²⁺, Mg²⁺, Zn²⁺), ammonia or organic amines (e.g. dicyclohexylamine, trimethylamine, triethylamine, pyridine, picoline, ethanolamine, diethanolamine, triethanolamine) or basic amino acids (e.g. arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

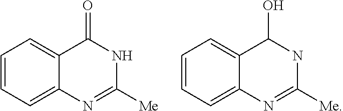

As another example, guanidines may exhibit the following isomeric forms in protic organic solution, also referred to as tautomers of each other:

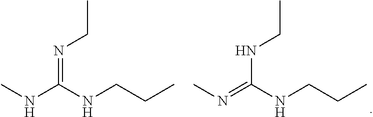

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

As used herein, the term “near infrared” (NIR) or “near infrared light” refers to light having a wavelength of about 700 nm to about 1400 nm.

As used herein, the term “visible light” refers to light having a wavelength of about 380 nm to about 700 nm.

The Present Technology

The present technology provides compositions and methods for obtaining an optoacoustic image, in particular compositions and nanoemulsions useful in generating an optoacoustic image as well as methods of generating an optoacoustic image.

In an aspect, a composition includes at least one contrast agent covalently linked to a targeting agent, where the at least one contrast agent includes a dark quencher, the targeting agent includes an antigen, an antigen-targeting ligand (such as an antibody), a receptor ligand, or an adhesion peptide, and the at least one contrast agent and targeting agent are covalently linked via formula (I),

X¹-L¹-X² (I),

where X¹ is an amino, amide, S, or O of the at least one contrast agent, where prior to the covalent link the moiety respectively was a NH, SH, or OH of the contrast agent; L¹ is C₁-C₁₂ alkylene, C₂-C₁₂ alkenylene, C₅-C₁₂ cycloalkylene, or C₆-C₁₂ arylene; and X² is an amino, amide, S, or O of the targeting agent, where prior to the covalent link the moiety respectively was a NH, SH, or OH of the targeting agent. In any embodiment herein, X¹ may be an amino, amide, thiol, hydroxyl, or the OH of a carboxylic acid prior to the covalent link. In any embodiment herein, X² may be an amino, amide, thiol, hydroxyl prior, or the OH of a carboxylic acid to the covalent link. In any embodiment herein, L¹ may be —C(O)—C₁-C₁₂ alkylene, —C₁-C₁₂ alkylene-C(O)—, —C(O)—C₁-C₁₂ alkylene-C(O)—, —C(O)—C₂-C₁₂ alkenylene, —C₂-C₁₂ alkenylene-C(O)—, —C(O)—C₂-C₁₂ alkenylene-C(O)—, —C(O)—C₅-C₁₂ cycloalkylene, —C₁-C₁₂ cycloalkylene-C(O)—, —C(O)—C₅-C₁₂ cycloalkylene-C(O)—, —C(O)—C₆-C₁₂ arylene, —C₆-C₁₂ arylene-C(O)—, or —C(O)—C₆-C₁₂ arylene —C(O)—.

The at least one contrast agent includes a dark quencher. The term “dark quencher” as used herein refers to a class of dyes that mainly emit absorbed energy (e.g., light energy with wavelengths shorter than about 700 nm) mainly as heat, by a non-radiative conversion of light energy accompanied by the formation of acoustic waves. As a result, the thermoelastic expansion is larger for quenchers than for light emitting fluorochromes. In any embodiment herein, the dark quencher may include, but is not limited to, a black hole quencher, dimethylaminoazobenzenesulfonic acid (Dabcyl), IRDye QC-1, DYQ-4, DYQ-700, Qx1 quencher, Iowa black FQ, Iowa black RQ, Atto 540Q, Atto 575Q, Atto 580Q, Atto 612Q, BHQ-0, BHQ-1, BHQ-2, BHQ-3, BHQ-10, BBQ-650, Ellipse, DYQ1, DYQ2, DYQ3, DYQ4, DYQ-425, DYQ505, DYQ700, DYQ660, DYQ661, or a combination of any two or more thereof. In certain embodiments, the dark quencher is a black hole quencher.

The composition may include a near infrared (NIR) fluorescent dye in any embodiment described herein. Exemplary NIR fluorescent dyes include, but are not limited to, cyanine 7 (Cy7) dye, cyanine 7.5 (Cy7.5) dye, IR780 dye, IR140 dye, Atto740 dye, DY-700 dye, DiR dye, indocyanine green (ICG), any fluorescent dye listed in Table 1 below, or a combination of any two or more thereof. In any embodiment herein, the NIR fluorescent dye may also be covalently linked to the targeting agent via a linker of formula (II)

X³-L²-X⁴ (II)

where X¹³ is a amino, S, or O of the NIR fluorescent dye, where prior to the covalent link the moiety respectively was a NH, SH, or OH of the NIR fluorescent dye; L² is C₁-C₁₂ alkylene, C₂-C₁₂ alkenylene, C₅-C₁₂ cycloalkylene, or C₆-C₁₂ arylene; and X⁴ is a amino, S, or O of the targeting agent, where prior to the covalent link the moiety respectively was a NH, SH, or OH of the targeting agent. In any embodiment herein, L² may be —C(O)—C₁-C₁₂ alkylene, —C₁-C₁₂ alkylene-C(O)—, —C(O)—C₁-C₁₂ alkylene-C(O)—, —C(O)—C₂-C₁₂ alkenylene, —C₂-C₁₂ alkenylene-C(O)—, —C(O)—C₂-C₁₂ alkenylene-C(O)—, —C(O)—C₅-C₁₂ cycloalkylene, —C₁-C₁₂ cycloalkylene-C(O)—, —C(O)—C₅-C₁₂ cycloalkylene-C(O)—, —C(O)—C₆-C₁₂ arylene, —C₆-C₁₂ arylene-C(O)—, or —C(O)—C₆-C₁₂ arylene —C(O)—. Alternatively, in any embodiment herein, it may be that the NIR fluorescent dye is not covalently linked to the targeting agent.

TABLE 1
Exemplary Contrast Agents
Dark Quenchers

| | |
|---|---|
| 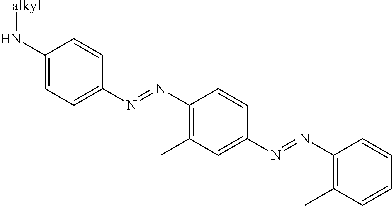 | BHQ-0 |
| 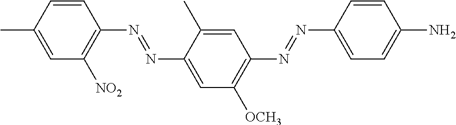 [SMILES: COc1cc(/N=N/c2ccc(C)cc2[N+](=O)[O-])c(C)cc1/N=N/c1ccc(N)cc1] | BHQ-1 |
| 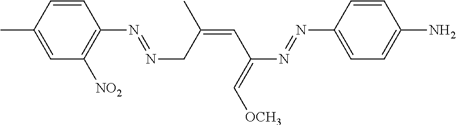 [SMILES: CO/C=C(/C=C(/C)C/N=N/c1ccc(C)cc1[N+](=O)[O-])\N=N\c1ccc(N)cc1] | BHQ-2 |
| 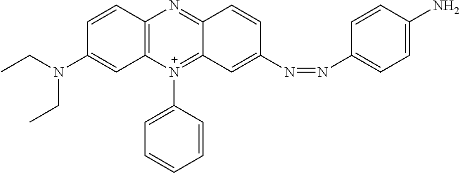 [SMILES: CCN(CC)c1ccc2nc3ccc(/N=N\c4ccc(N)cc4)cc3[n+](-c3ccccc3)c2c1] | BHQ-3 |
| 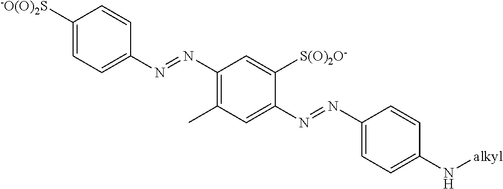 | BHQ-10 |
| 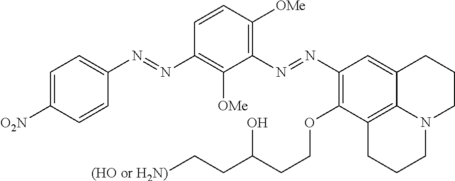 | BBQ-650 |
| 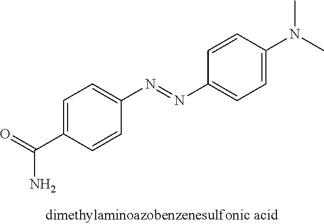 [SMILES: CN(C)c1ccc(/N=N/c2ccc(C(N)=O)cc2)cc1] | Dabcyl |
| 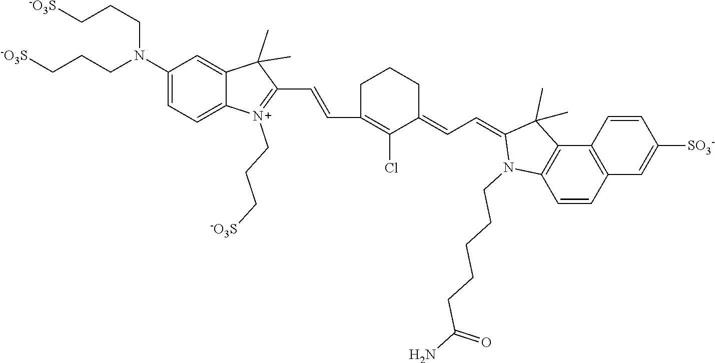 | IRDye ® QC-1 |
| 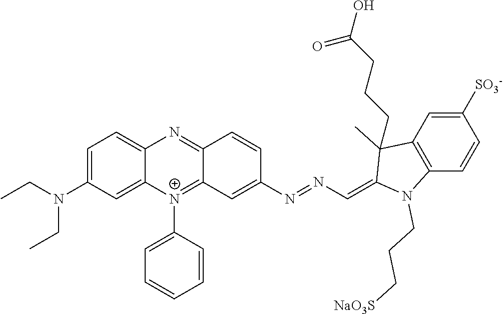 | DYQ-700 |
| 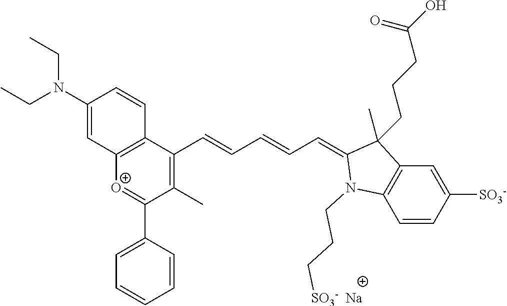 | DYQ-4 |

Fluorescent Dyes

| | |
|---|---|
| 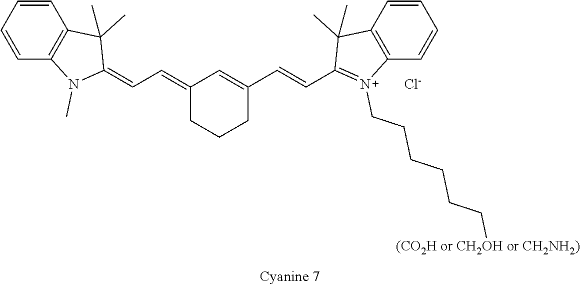 | Cy7 |
| 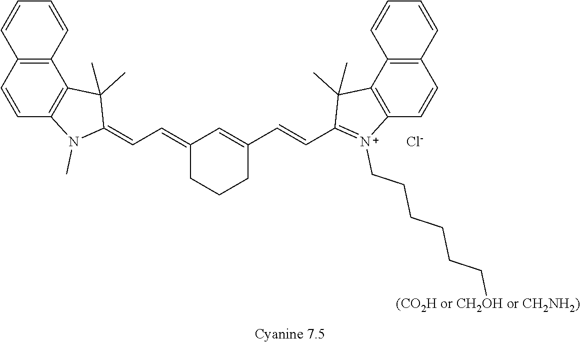 | Cy7.5 |
| 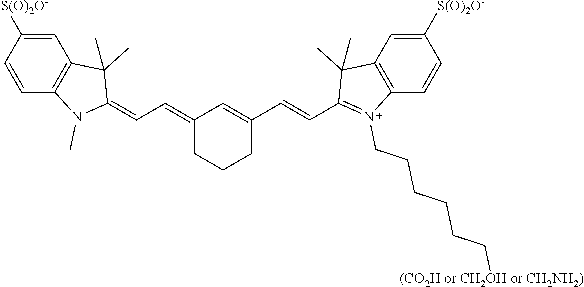 | sulfo-Cy5 |
| 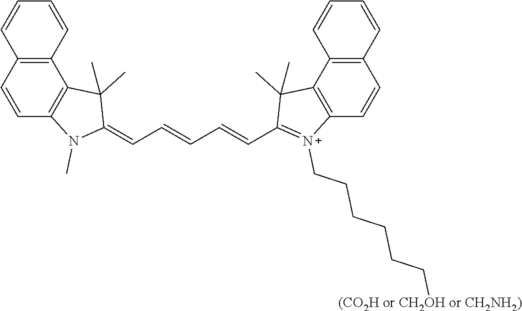 | Cy5.5 |
| 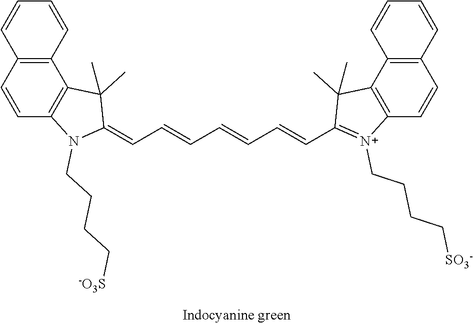 | ICG |
| 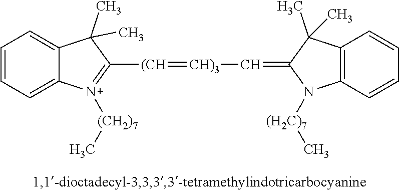 | DiR |
| 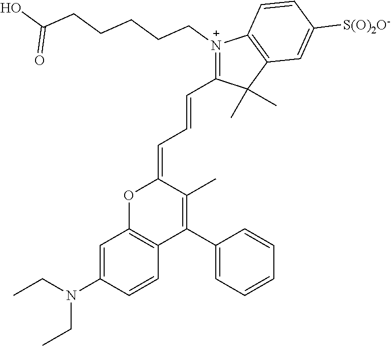 | DY-700 |
| 2-[2-[2-chloro-3-[(1,3-dihydro-3,3-dimethyl-1-propyl-2H- | IR780 |
| indol-2-ylidene)ethylidene]-1-cyclohexen-1-yl]ethenyl]-3,3- | |
| dimethyl-1-propylindolium halide | |
| 5,5′-dichloro-11-diphenylamino-3,3′-diethyl-10,12- | IR140 |
| ethylenethiatricarbocyanine perchlorate | |

The targeting agent may be an antibody, an antigen, an antigen-targeting ligand, a receptor ligand, an adhesion peptide, or a combination of any two or more thereof. Suitable adhesion peptides include, but are not limited to, a RGD peptide, a cRGD peptide, and heparin-binding peptides. Suitable antigen-targeting ligands include, but are not limited to, prostate-specific membrane antigen (“PSMA”) ligands, Trastuzumab, A33, 5B1, M9364A, exendin-4, bombesin, minigastrin (CCK-2R), and folate. For example, PSMA ligands include

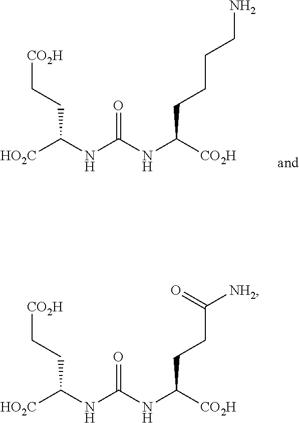 [SMILES: NCCCC[C@H](NC(=O)N[C@@H](CCC(=O)O)C(=O)O)C(=O)O ; NC(=O)CC[C@H](NC(=O)N[C@@H](CCC(=O)O)C(=O)O)C(=O)O]

where a composition according to the present technology may be of the following structures

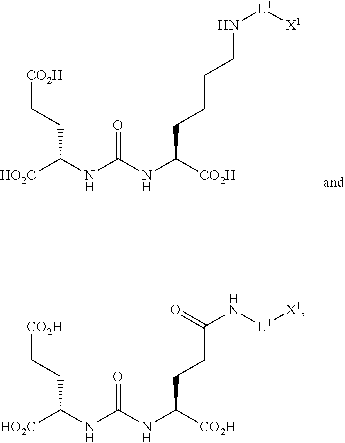

respectively.

In an aspect, the present technology provides an oil-in-water nanoemulsion composition that includes a plurality of lipid micelles in an aqueous phase. Each lipid micelle includes an interior and an exterior of the micelle; an outer amphiphilic component in contact with the aqueous phase on the exterior and with an oil phase on the interior; and at least one contrast agent within the interior. The plurality of lipid micelles exhibit an intensity-weighted average particle diameter of about 100 nm to about 500 nm as determined by dynamic light scattering. The intensity-weighted average particle diameter may be about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, about 300 nm, about 350 nm, about 400 nm, about 450 nm, about 500 nm, or any range including and/or in between any two of these values. For example, in any embodiment herein the intensity-weighted average particle diameter may be about 120 nm to about 150 nm. A weight ratio of amphiphilic component to oil phase may be from about 0.1:1, about 0.2:1, about 0.3:1, about 0.4:1, about 0.5:1, about 0.6:1, about 0.7:1, about 0.8:1, about 0.8:1, about 1:1, or any range including and/or in between any two of these values. A weight ratio of oil phase to contrast agent may be about 1:0.001, about 1:0.002, about 1:0.003, about 1:0.004, about 1:0.005, about 1:0.006, about 1:0.007, about 1:0.008, about 1:0.009, about 1:0.01, about 0:0.02, about 1:0.03, about 1:0.04, about 1:0.05, about 1:0.06, about 1:0.07, about 1:0.08, about 1:0.09, about 1:0.1, or any range including and/or in between any two of these values. For example, the weight ratio of amphiphilic component:oil phase: contrast agent may be 0.5:1:0.04.

The amphiphilic component may include fatty acids, phospholipids, pegylated phospholipids, and combinations of any two or more thereof. For example, the phospholipid may include 1,2-diestearoyl-sn-glycero-3-phosphocholine (DSPC), pegylated 1,2-distearoyl-sn-glycero-3-phosphoethanolamine (DSPE), 1,2-dipalmitoyl-sn-glycero-3-phosphocholine (DPPC), 1,2-distearoyl-sn-glycero-3-phosphoethanolamine-N-[amino(polyethylene glycol)-2000] (ammonium salt) (DSPE-PEG2000-amine), 1,2-distearoyl-sn-glycero-3-phosphoethanolamine-N-[folate(polyethylene glycol)-2000] (ammonium salt) (DSPE-PEG2000-folate), cholesterine, or combinations of any two or more thereof.

The nanoemulsion composition includes at least one contrast agent. Suitable contrast agents include, but are not limited to, dark quenchers, fluorescent dyes, and combinations thereof. Suitable dark quenchers and fluorescent dyes are discussed previously. Preferably, the nanoemulsions include both a dark quencher and a fluorescent dye, either as independent molecules or according to any embodiment of the compositions of the present technology as described herein that include both a dark quencher and a fluorescent dye.

The nanoemulsion composition of the present technology includes an aqueous phase that includes water. The aqueous phase may include one or more of a phosphate buffer, HEPES, ethanol, and chloroform. In any embodiment herein, the aqueous phase may include a phosphate buffer solution.

The nanoemulsion composition of the present technology includes an oil phase. The oil of the oil phase includes at least one of a medium chain triglyceride (where “medium chain” means C₈ or C₁₀ fatty acids of the triglyceride; e.g. MIGLYOL 810, MIGLYOL 812), peanut oil, sesame oil, soybean oil, cottonseed oil, corn oil, olive oil, ethyl oleate, isopropyl myristate, fatty acid glycerides, and acetylated fatty acid glycerides.

In a related aspect of the present technology, a method is provided for obtaining an optoacoustic image of a subject. The method includes contacting a target with a composition and/or nanoemulsion described herein in any embodiment; contacting the target with light, where the light has a wavelength in the visible to near infrared spectrum; and detecting an optoacoustic signal from the target.

The target may include a tumor, where the tumor includes tumor cells. Exemplary tumor cells include, but are not limited to, endothelial cells, epithelial cells, glioblastoma cells, breast cancer cells, pancreatic cancer cells, bladder cancer cells, or a combination of any two or more thereof. In some embodiments, the tumor cells express αᵥβ₃-integrin.

The method of obtaining an optoacoustic image includes contacting the target with light, where the light has a wavelength in the visible to near infrared spectrum. The light may a wavelength of about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, or any range including and/or in between any two of these values.

In another related aspect, a method is provided for obtaining an optoacoustic image in a subject. The method includes administering to the subject an effective amount of a composition and/or nanoemulsion described herein; contacting a target area of the subject with light, wherein the light has a wavelength in the visible to near infrared spectrum; and detecting an optoacoustic signal (i.e., ultrasound waves) from the target area of the subject.

In an aspect of the present technology, a composition is provided that includes any one of the aspects and embodiments of compositions and/or nanoemulsions of the present technology and a pharmaceutically acceptable carrier. As used herein, a “pharmaceutically acceptable carrier” includes carriers and/or excipients. In a related aspect, a pharmaceutical composition is provided, the pharmaceutical composition including an effective amount of the composition and/or nanoemulsion for imaging a condition; and where the condition includes a tumor. In a further related aspect, an imaging method is provided that includes administering a composition and/or nanoemulsion of any embodiment described herein or administering a pharmaceutical composition comprising an effective amount of a composition and/or nanoemulsion of any embodiment described herein to a subject and, subsequent to the administering, detecting ultrasound waves. In any embodiment of the imaging method, the subject may be suspected of suffering from a condition that includes a mammalian tissue overexpressing PSMA, such as a cancer expressing PSMA (including cancer tissues, cancer related neo-vasculature, or a combination thereof), pancreatic cancer such as insulinoma, diabetes, and bladder cancer. The cancer of the pharmaceutical composition and/or the method may include one or more of glioma, cervical carcinoma, vulvar carcinoma, endometrial carcinoma, primary ovarian carcinoma, metastatic ovarian carcinoma, non-small cell lung cancer, small cell lung cancer, bladder cancer, colon cancer, primary, gastric adenocarcinoma, primary colorectal adenocarcinoma, renal cell carcinoma, and prostate cancer.

“Effective amount” refers to the amount of a compound or composition required to produce a desired effect, such as a quantity of a compound of the present technology necessary to be detected by the detection method chosen. For example, an effective amount of a compound of the present technology includes an amount sufficient to enable detection of binding of the compound to a target of interest including, but not limited to, one or more of glioma, cervical carcinoma, vulvar carcinoma, endometrial carcinoma, primary ovarian carcinoma, metastatic ovarian carcinoma, non-small cell lung cancer, small cell lung cancer, bladder cancer, colon cancer, primary, gastric adenocarcinoma, primary colorectal adenocarcinoma, renal cell carcinoma, and prostate cancer (such as castration resistant prostate cancer). Another example of an effective amount includes amounts or dosages that are capable of providing a detectable optoacoustic image (above background) in a subject with, e.g., a tissue overexpressing PSMA, such as, for example, statistically significant emission above background. As used herein, a “subject” or “patient” is a mammal, such as a cat, dog, rodent or primate. Typically the subject is a human, and, preferably, a human suffering from or suspected of suffering from one or more of the conditions previously described. The term “subject” and “patient” may be used interchangeably.

The instant present technology provides pharmaceutical compositions and medicaments comprising a composition and/or nanoemulsion of any embodiment described herein and a pharmaceutically acceptable carrier or one or more excipients or fillers (collectively, such carriers, excipients, fillers, etc., will be referred to as “pharmaceutically acceptable carriers” unless a more specific term is used). The compositions may be used in the methods and imagings described herein. Such compositions and medicaments include an effective amount of any compound as described herein, including but not limited to a composition and/or nanoemulsion of any embodiment described herein, for imaging one or more of the herein-described conditions. The pharmaceutical composition may be packaged in unit dosage form. For example, the unit dosage form is effective in imaging a mammalian tissue overexpressing PSMA, such as a cancer expressing PSMA (including cancer tissues, cancer related neo-vasculature, or a combination thereof), when administered to a subject.

The pharmaceutical compositions and medicaments may be prepared by mixing one or more a composition and/or nanoemulsion of any embodiment described herein, with pharmaceutically acceptable carriers, excipients, binders, diluents or the like to image the herein-described disorders. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more a composition and/or nanoemulsion of the instant present technology with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. An isotonic solution will be understood as isotonic with the subject. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

A composition and/or nanoemulsion of any embodiment described herein of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of a composition and/or nanoemulsion of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles (such as the nanoemulsions of the present technology) or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts until, for example, statistically significant resolution (via, e.g., optoacoustic imaging) is achieved. The compounds of the present technology may be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition being imaged and the pharmacological activity of the compound being used. The determination of optimum dosages for a particular patient is well known to those skilled in the art. Various assays and model systems can be readily employed to determine the effectiveness of a compound according to the present technology.

The compounds of the present technology can also be administered to a patient along with other conventional imaging agents that may be useful in the imaging of a condition and/or disorder. Thus, a pharmaceutical composition of the present technology may further include an imaging agent different than a composition and/or nanoemulsion of any embodiment described herein. The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology may also include administering, either sequentially or in combination with one or more compounds of the present technology, a conventional imaging agent in an amount that can potentially or synergistically be effective for the imaging of a mammalian tissue overexpressing PSMA.

In an aspect, a compound of the present technology is administered to a patient in an amount or dosage suitable for imaging. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

The examples herein are provided to illustrate advantages of the present technology and to further assist a person of ordinary skill in the art with preparing or using the compounds of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### EXAMPLES

Cell Lines and Animals

Human glioblastoma cells (U-87 MG) expressing α₃βᵥ-integrin were obtained from ATCC® (VA, US) and cultured in Eagle's Minimum Essential Medium (Corning Cellgro, VA, US) containing 10% FBS, 1% Penicillin/Streptavidin solution, 2 mM L-glutamine, 1 mM sodium pyruvate and 0.075% (w/v) sodium bicarbonate. The cells were incubated in a humidified 5% CO₂ atmosphere.

For in vivo experiments, 6-8 week old female Hsd: Athymic Nude-Foxn1^{nu} mice were purchased from Envigo (IN, US). All animal experiments were performed in accordance with institutional guidelines and approved by the IACUC of MSK, and followed NIH guidelines for animal welfare.

### Example 1: Synthesis and Characterization of cRGD-Labeled Black Hole Quencher (BHQ-1-cRGD)

To a stirring solution of cyclic RGD (cyclo(Arg-Gly-Asp-D-Phe-Lys) (cRGD), 1.0 mg, 1.66 μmol, 1.0 eq., Peptides International, KY, US) in anhydrous DMF (200 μL) a solution of BHQ-1 N-hydroxysuccinimide (NHS) ester (1.2 mg, 2.0 μmol, 1.2 eq., Biosearch Technologies, CA, US) in anhydrous DMF (200 μL) and trimethylamine (1.2 μL, 8.0 μmol, 4.8 eq.) was added sequentially. The resulting reaction solution was stirred for 4 h at room temperature. Purification by high performance liquid chromatography (HPLC) (C18, 10% to 100% of acetonitrile over 10 min, then 100% of acetonitrile until 18 min, 1.0 mL/min) yielded BHQ-1-cRGD (1.5 mg, 1.4 μmol, 82%) as a black solid. The purity of BHQ-1-cRGD was analyzed by performing analytical HPLC (rₜ=11.0 min, >97%). The identity of the product (C₃₅H₆₇N₁₅O₁₁, MW: 1090.21 g/mol) was verified by electrospray ionization mass spectrometry (ESI(+): m/z (%) 546.70 (100) [M+2H]²⁺, 1090.40 (25) [M+H]⁺). The absorbance spectra of BHQ-1 and BHQ-1-cRGD were measured in ethanol using spectrophotometry from 250 nm to 750 nm.

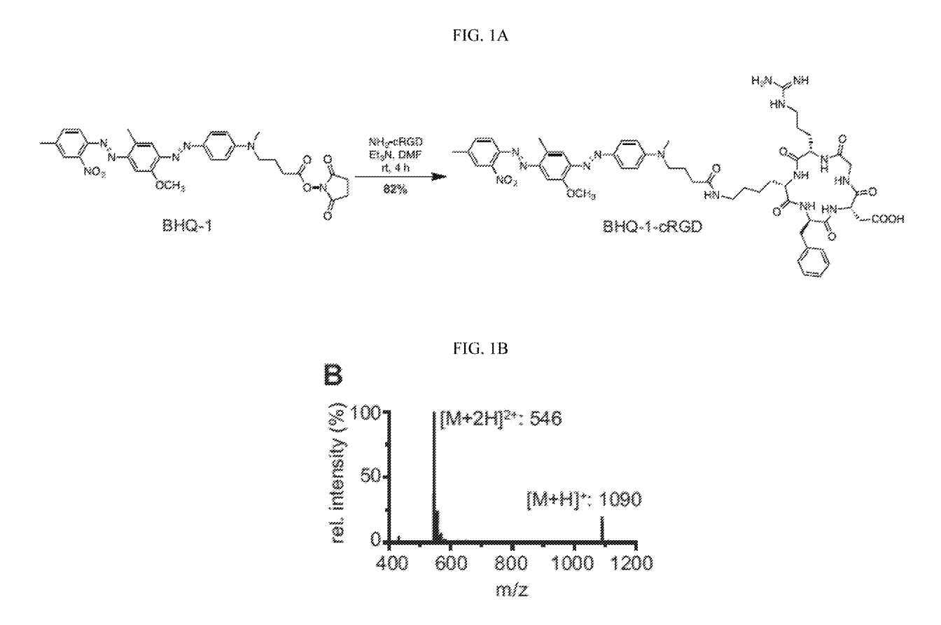

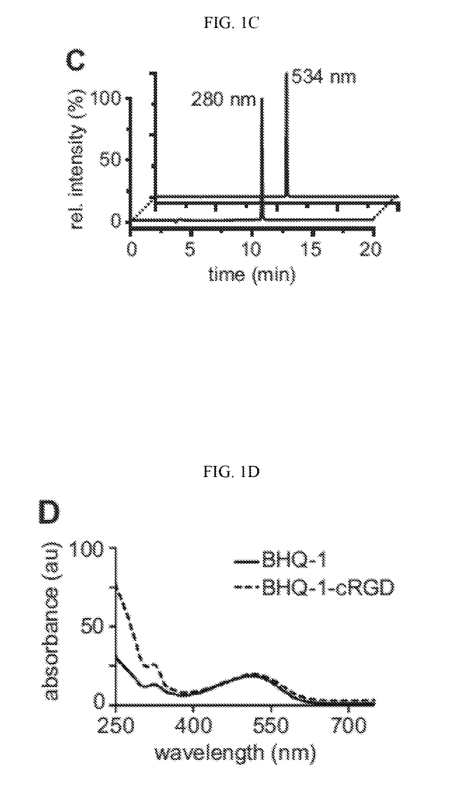

Synthesis of the black hole quencher (BHQ-1) labeled adhesion peptide (cRGD) is shown in FIG. 1A. In the presence of trimethylamine, the NHS ester-activated BHQ-1 could be successfully conjugated to the primary amine of the cRGD and the final product was isolated in 82% yield after HPLC purification. Identity and purify of BHQ-1-cRGD were validated successfully through mass spectrometry and analytical HPLC resulting in a molecular weight of 1,090 g/mol (FIGS. 1B and 1C). The absorption spectrum of unlabeled BHQ-1 and BHQ-1-cRGD peaked at 515 nm, suggesting that the labeling procedure did not change the optical properties of the black quencher (FIG. 1D). Additionally, the spectrum of BHQ-1-cRGD showed another peak at 280 nm, representing the RGD peptide in the probe.

### Example 2: In Vitro, In Vivo, and Ex Vivo Optoacoustic Imaging BHQ-1-cRGD

Optoacoustic Imaging

For imaging, a high-resolution raster-scan optoacoustic mesoscopy (RSOM) prototype scanner was used in an epi-illumination mode. The scanner illuminates the tissue with a fast monochromatic nanosecond laser (1 ns, 2 kHz, 1 mJ pulse energy at 532 nm). The laser light was coupled to the sample using a three arm fiber bundle which is combined with the ultrasound detector into a single scan unit. The optoacoustic signals were measured with a 50 MHz spherically focused detector and a bandwidth of 5-80 MHz. Furthermore, the signals were amplified with a 63 dB low noise amplifier and digitized using a fast 12 bit data acquisition card. The scan was performed in a continuous-discrete manner. The usual scan took 1.30 minutes for a field of view of 8×8 mm² and the maximum depth was about 2 mm, limited by the penetration depth of 532 nm photons in tissue.

The raw signals were transformed to the computer on which they were later reconstructed using beam forming. Before reconstruction, the signals were divided into two sub-bands: low frequencies 5-25 MHz and high frequencies 25-80 MHz. These sub-bands were later on separately reconstructed an overlayed using different colors (red=low frequencies, green=high frequencies). For the reconstruction a speed of sound of 1540 m/s has been used and the reconstruction has been performed with voxel sizes of 20×20×5 μm³. Finally, the Hilbert transform was applied to the reconstructed volume to remove the negative values and the images were further processed using a Wiener and a Median filter to improve the signal to noise ratio. For visualization we took the maximum intensity of the three-dimensional volume.

Agarose Phantom Studies of BHQ-1

the quencher dye BHQ-1 (10 mM in DMSO) was embedded at different concentrations (0 μM to 500 μM) into 1% agarose phantoms (10 μl, preheated at 70° C.). Agarose drops containing the agent were placed next to each other into a 1% agarose-coated petri dish and covered with water to enable ultrasound signal detection. The experiment was done in triplicates and RSOM was performed as described under 2.3 but without dividing the detected ultrasound frequencies into smaller sub-bands during the reconstruction process. The generated images were analyzed using ImageJ and regions of interest (ROIs) were drawn over the phantoms to quantify the detected optoacoustic signals of the BHQ-1 dilution series.

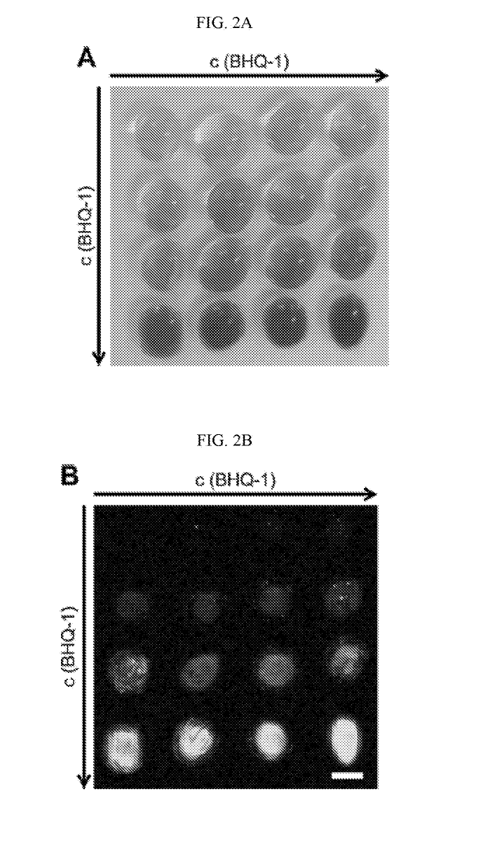

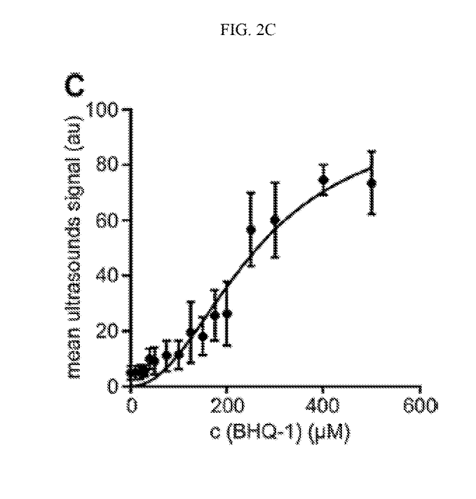

A visible change I color of the phantoms was detected from transparent to purple with increasing concentration of BHQ-1 from 0 μM to 500 μM (FIG. 2A). Furthermore, an increasing optoacoustic signal was detected with increasing concentration of BHQ-1 using RSOM (FIG. 2B). Stronger signals are reflected in a more intense green compared to lower signals. The quantitative analysis of the RSOM image of the BHQ-1 phantoms confirmed the optical findings and revealed increasing mean optoacoustic signals from 4.8±2.4 AU (arbitrary units) out of the 0 μM phantom up to 73.4±11.2 AU from the 500 μM phantom (FIG. 2C)

RSOM of BHQ-1-cRGD in U-87 Cell Spheroids In Vitro

U-87 cell spheroids were grown by transferring 1×10⁶ U-87 cells, raised in normal growth medium, into a 1% agarose-coated cell culture flask (75 cm²) and incubated for about one week. After they reached sizes of approximately 0.6 to 1.0 mm, 12 spheroids were collected and transferred into agarose-coated wells of a 96-well plate. Representative images of the spheroids were taken using a light microscope. For 2 hours, 4 spheroids were incubated with 50 μl cell culture medium without the probe, with 50 μM BHQ-1-cRGD or with 50 μM BHQ-1-cRGD and a 100-fold excess (5 mM) of free cyclic RGD, respectively. Afterwards, the spheroids were gently washed for three times with phosphate buffer solution (PBS), embedded in 1% agarose in a petri dish and covered with water. After performing RSOM as described under 2.3 without dividing the ultrasound frequencies into smaller sub-bands during the reconstruction process, the spheroid images were analyzed quantitatively using ROI measurement in ImageJ and calculating the signal to noise ratio of each spheroid.

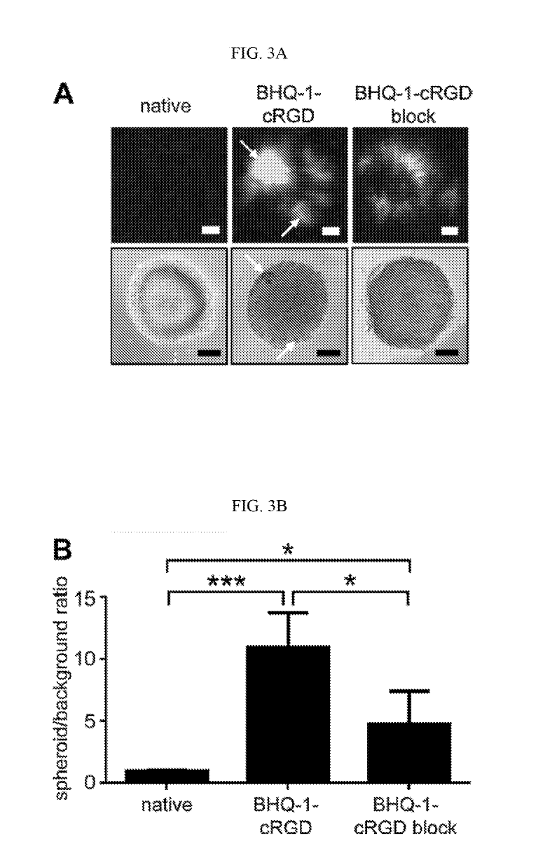

After 2 h, a strong optoacoustic signal could not be detected following incubation of U-87 glioblastoma cell spheroids with the BHQ-1-cRGD probe (FIG. 3A). Almost no signal appeared in the native spheroids without the probe and a comparatively low optoacoustic signal was depicted after incubation with a 100-fold excess of free cyclic RGD simultaneously to the BHQ-1-cRGD probe. A comparison of the RSOM images with the white light pictures highlighted that a high binding BHQ-1-cRGD onto cells results in a stronger optoacoustic signal. The quantitative analysis confirmed the successful binding of BHQ-1-cRGD onto u-87 cells at a concentration of 50 μM with a signal to noise ratio of 11.0±2.8 (FIG. 3B). In comparison, the native spheroids themselves did not generate an optoacoustic signal above background noise with a statistically significant lower ratio of 1.0±0.0 (P<0.0005). Furthermore, the 100-fold excess of cyclic RGD blocked the binding of BHQ-1-cRGD to the spheroids significantly with a signal to noise ratio of only 4.8±2.7 (P≤0.05).

In Vivo Optoacoustic Imaging of BHQ-1 in Mouse Footpad

All mice were anesthetized using 2% isoflurane. For imaging the quencher dye in vivo, 10 nmol BHQ-1 solution were injected subcutaneously into the footpad of 3 mice. The mouse was then placed in a pre-warmed water bath on a mouse bed keeping the head of the animal above water level. The foot of the mouse was stabilized under water with tape to keep it in a static position for the measurement procedure. The feet were measured before and directly after injection using RSOM as described under 2.3. Quantitative analysis was done in ImageJ by placing a ROI over the injection site of each foot as well as over the background blood vessels in the foot pad.

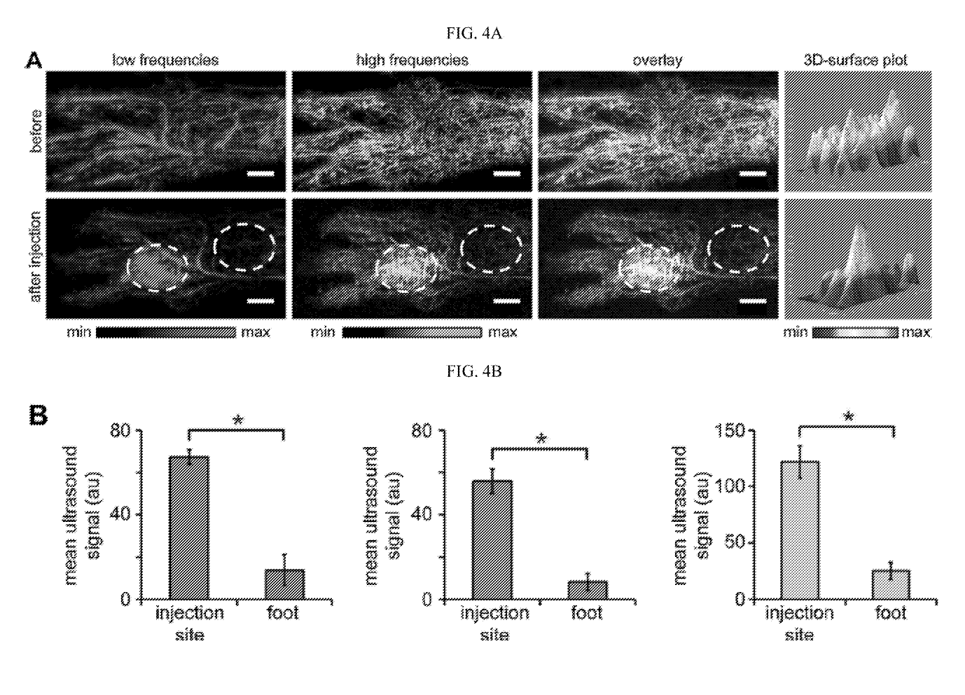

Following a subcutaneous injection of 10 nmol BHQ-1 into the mouse footpad, a strong optoacoustic signal could be detected at the injection site compared to the homogeneous signal before the application (FIG. 4A). At the same time, the relative intensity of the signal generated by hemoglobin in the blood vessels of the footpad decreased rapidly compared to before the injection. A stronger optoacoustic signal could be seen for the low as well as for the high frequencies and thus also for the overlay image of all frequencies. A 3D-surface plot of the footpad before and after injection confirmed these observations (FIG. 4A). The quantitative analysis revealed an almost 5-times higher optoacoustic signal at the injection site with 121.6±14.2 AU compared to the surrounding footpad vessels with only 25.2±7.7 AU for the overlay image of all detected frequencies (P≤0.0005, FIG. 4B, yellow). The same trend was observed for the separated low frequencies (5-25 MHz, red) with values of 67.2±3.4 AU versus 13.9±7.2 AU and the high frequencies (25-80 MHz, green) with 55.6±5.7 AU versus 8.3±3.9 AU respectively (P≤0.0005).

RSOM of BHQ-1-cRGD in U-87 Tumors Ex Vivo

U-87 tumors were implanted into 6 mice by injecting 2×10⁶ U-87 cells in 100 μl 1:1 matrigel:medium subcutaneously into the lower left back. After tumors reached a diameter of approximately 6 mm, one group of 3 mice were injected intravenously with 50 nmol BHQ-1-cRGD in 150 μl sodium chloride and a second group of 3 mice with 150 μl sodium chloride only as a control. After 2 h, mice were anesthetized using 2% isoflurane and perfused via the heart using PBS until all blood was removed from the body. The tumors were excised, placed in ultrasound gel in a petri dish, covered with a transparent foil and with water and imaged using RSOM as described under 2.3. Analysis was done using ImageJ by generating a profile plot for one representative tumor of each animal group.

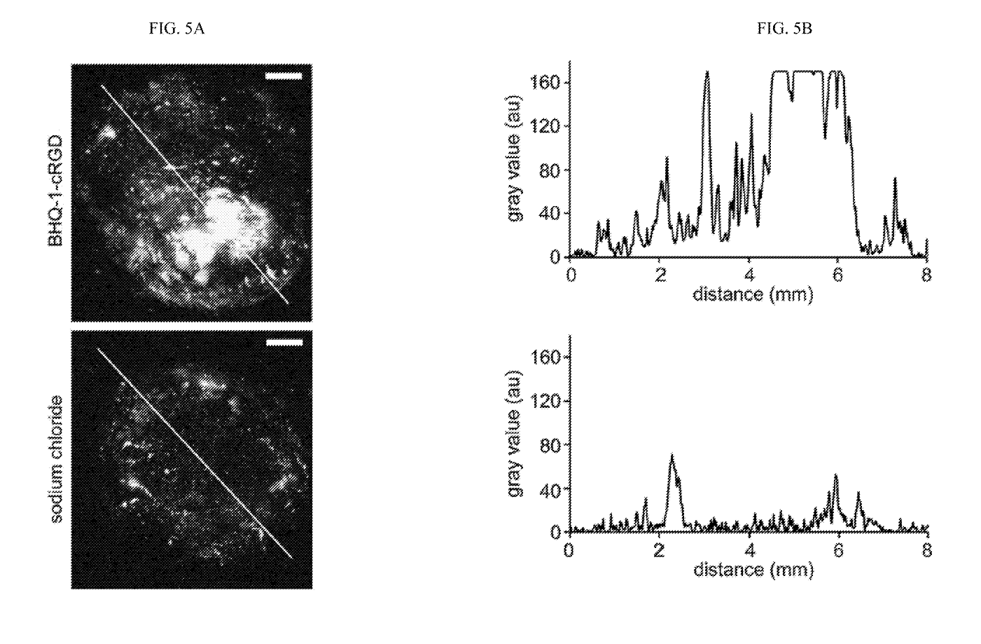

After intravenous injection of 50 nmol BHQ-1-cRGD into 3 mice followed by perfusion, a clear optoacoustic signal could be detected in these tumors compared to the ones with only sodium chloride as a control (FIG. 5A). The representative tumors show a clear trend for the specific accumulation of our probe in the U-87 tumors. The poor signal in the sodium chloride tumors indicates the effective wash out of the blood by the perfusion. By drawing a line through the tumors, a profile plot of the gray values could be generated, showing much higher optoacoustic values in the BHQ-1-cRGD injected mouse compared to the sodium chloride injected control animal (FIG. 5B). The strong signals in the BHQ-1-cRGD tumor show saturated values in some areas, meaning that the generated optoacoustic signals in these regions are extremely strong.

The above results illustrates the compositions of the present technology, including BHQ-1-cRGD, allow for high suitability for optoacoustic imaging in vitro as well as in vivo with the generation of strong optoacoustic signals. Furthermore, the results illustrate the compositions of the present technology provide tumor specific contrast agents for optoacoustic imaging.

### Example 3: Preparation and Characterization of Nanoemulsions

Optoacoustic Imaging

Spatial reconstruction of the data was performed using the ViewMSOT software suite (V3.6; iThera Medical) and a back-projection algorithm. The data were then transferred to MATLAB (R2017b) and subsequent analysis was performed using a GUI developed in house. The normalized optoacoustic reference spectra were obtained from optoacoustic intensities obtained from phantom scans. Scans were performed from 680-900 nm with 10 nm steps and the spectra were normalized to their respective optoacoustic signal maxima. To generate the DCLS models for in vivo, ex vivo, and in vitro studies, the reference optoacoustic spectra of the nanoemulsion compositions were used. For the investigation of spectral unmixing, the analysis was performed as follows: DCLS by using a Moore-Penrose pseudo-inverse matrix of the reference spectra; NN-LS with the PLS Toolbox v.8.0 (Eigenvector Research, Inc., Wenatchee, Wash., USA); PCA using the PCA function in MATLAB; and ICA using the fastica package in Matlab. The three-dimensional (3D) image reconstructions of the phantoms were shown produced with 50 surfaces, and 1 alpha. Quantitative image processing of the data was performed by defining the region of interest within a 2D slice (n=3) MSOT image.

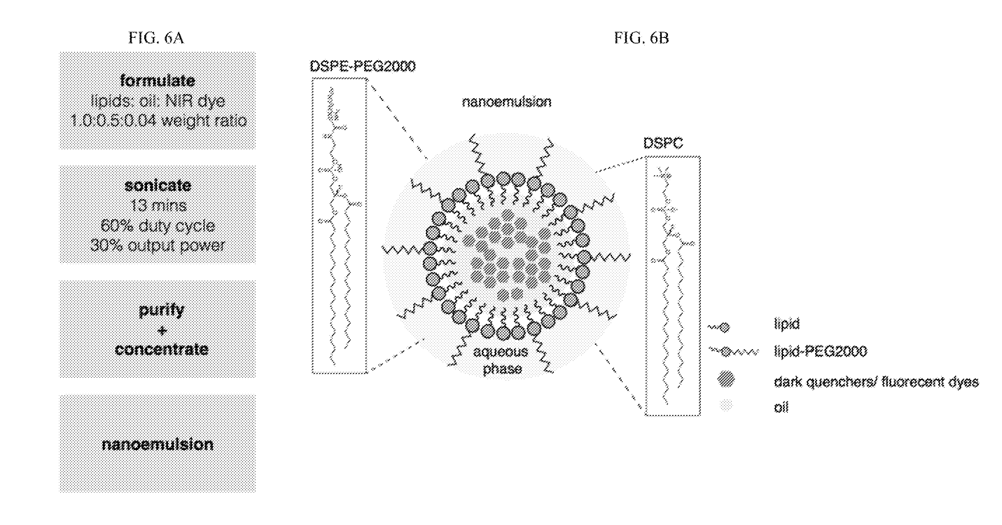

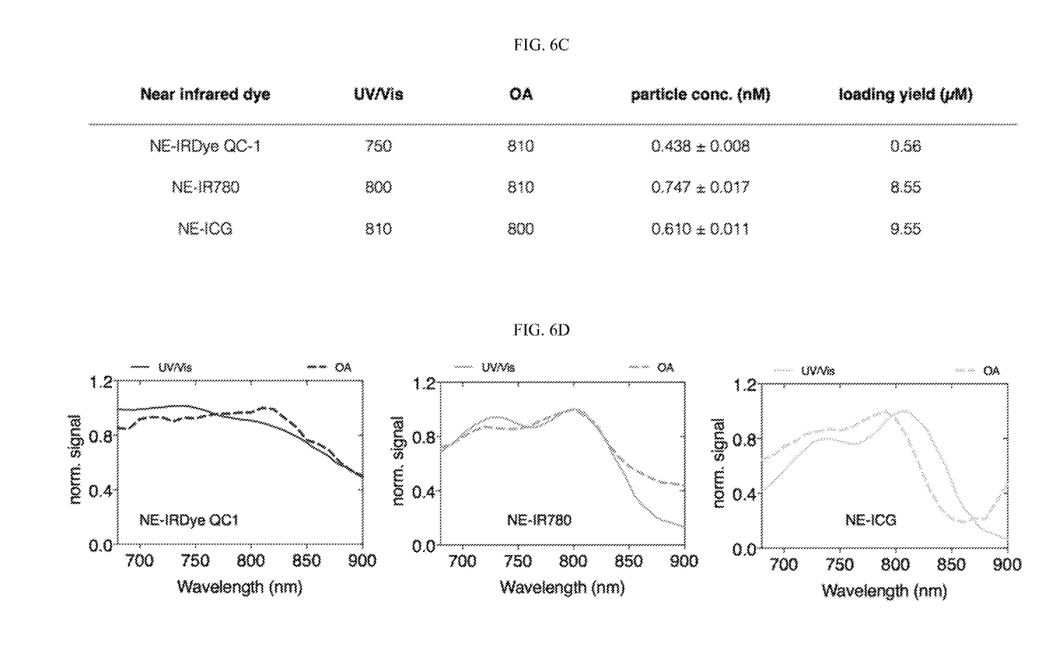

The nanoemulsion compositions according to the present technology were formulated via solvent displacement, as shown in FIG. 6A. A lipid stock solution composed of 1,2-diesteroyl-sn-glycero-3-phosphocholine (DSPC), pegylated DSPE (DSPE-PEG2000) and cholesterol in a 62:5:33 molar ratio was prepared in EtOH (25 mg/mL). For all phantom preparations and starting with 130 μM of dye, non-functionalized nanoemulsion composed of lipids, medium chain triglycerides (“MCT”; Miglyol® 812 N, Oleochemicals, IOI group GmbH, Germany) and near-infrared dye in a 0.5:1:0.01 weight ratio were mixed together. The oil and the dye were mixed together first. Volumes of the resultant mixtures were made up to a 1000 μL (EtOH). Via a solvent displacement and diffusion method, the nanoemulsion was obtained by swiftly injecting 1 mL of the ethanolic mixture of oil and dye into 20 mL of PBS, immersed an ultrasonication cold bath. Ultrasonication was continued until the nanoemulsions reached a desired droplet size or a constant nanoemulsion size was observed. The nanoemulsions were purified using a multi-step process. First, the nanoemulsions were subjected to centrifugation at 4000 rpm (22° C.) for 30 mins to remove possible aggregates. A KrosFlo® MiniKros Pilot filtration system (KMPi TFF system) fitted with a mPES MicroKross® modules 100 kDa MWCO was used to concentrate down to a total volume of 2000 μL. If necessary, a 100 kDa MWCO centrifugal viva spin filter was used for further washing steps and reducing volumes. The formulation was passed through a PES syringe filter (0.22 μm, 13 mm diameter, Celltreat Scientific Products, Pepperell, Mass.) before characterization and/or administration. For in vivo studies, nanoemulsion preparations were formulated with lipids, MCT (Miglyol® 812 N), and near-infrared dye in a 0.5:1:0.04 weight ratio. For in vivo, the starting dye concentration was 520 μM.

To assess optoacoustic detection of the nanoemulsion composition spectra under controlled conditions, we imaged nanoemulsion samples embedded in a cylindrical, light-scattering phantom. For simulating the tissue background in biological systems, soft tissue mimicking phantoms were fabricated by combining two methods to produce an acoustic attenuation of 0.495 dB/cm/MHz, all according the generic tissue definition given by Cook et. al. Tissue-mimicking phantoms for photoacoustic and ultrasonic imaging. Biomedical Optics Express, 2011. 2(11): p. 3193-3206. Specifically, soft tissue mimicking phantoms were freshly prepared by adding 15% v/v Intralipid® 20%, I.V. fat emulsion to provide the scattering and 0.01 mM Direct Red 81 for absorption to a pre-warmed solution of 1.5% v/v agarose Type 1 (solid in <37° C.) in Milli Q water (18.2 MΩ-cm at 25° C.). The solution was poured into a 20 mL syringe (2 cm diameter) serving as a plastic mold to create a cylindrical shape of the phantom, into which a sealed thin walled optically clear plastic straw containing the nanoemulsion or contrast agent of interest was inserted. To compare their relative optoacoustic imaging potential, three serial dilutions of the nanoemulsions were prepared: high (1×), medium (½×) and low (¼×). The phantom was allowed to cool at room temperature until the agarose solidified. Clinically relevant and commercially available contrast agents (IR780, IR140, Atto740, IRDye QC-1, DY831, DYQ4, Cy7, Cy7.5, DY700, DYQ700, and DiR) were prepared and measured in DMSO and their corresponding nanoemulsions were measured in phosphate buffered saline for the MSOT phantom studies.

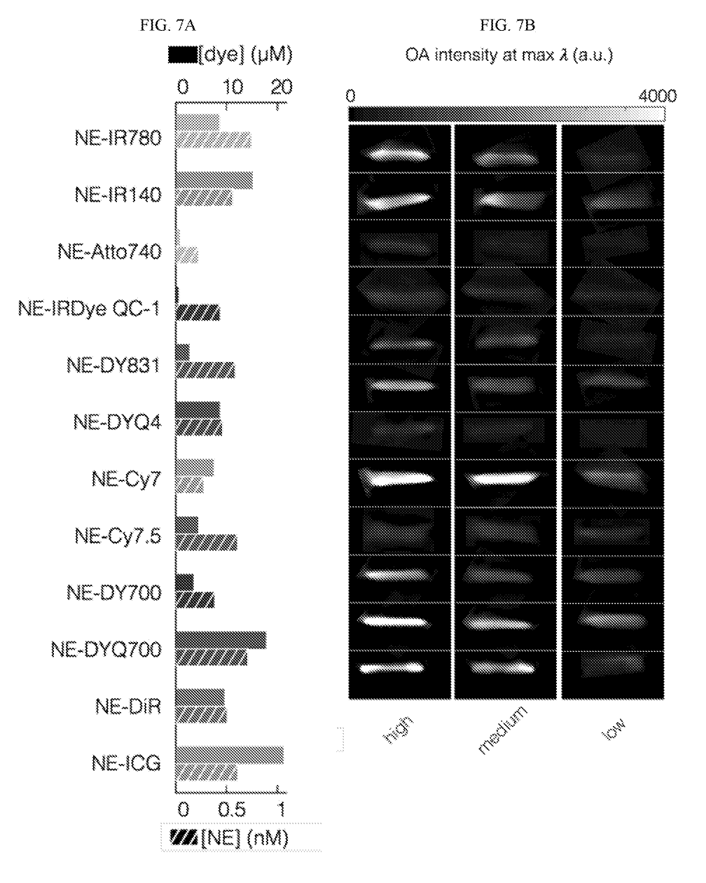

The nanoemulsion compositions had a minimum effective diameter of 119 nm±8.5 nm and a maximum effective diameter of 149 nm±5.8 nm with particle concentrations ranging from 0.23 to 0.75 nM, zeta potentials between −0.24 and −6.12 mV and PDI≤0.15. The yield and amount of contrast agent encapsulated for selected nanoemulsion compositions are shown in FIG. 7A. For MSOT interrogation of the nanoemulsion compositions, we prepared tissue mimicking phantoms. The phantoms featured a series of pouches, enclosing a serial dilution of a contrast agent (dye or NE concentrations were tested: high (1×), medium (½×) and low (¼×) concentration), as well as a pouch filled with ICG (3 μM in DMSO) as an internal standard. Shown in FIG. 6D, the recorded optoacoustic spectra of selected nanoemulsions were found to slightly deviate from their UV/Vis spectra.

### Example 4: Ex Vivo and In Vivo Optoacoustic Imaging Nanoemulsion Compositions

Ex Vivo Optoacoustic Imaging

In ex vivo MSOT experiments, a total of seven female homozygous Hsd:athymic mice Nude-Foxn1ⁿ(6-8 weeks old) were used for each cohort of experiment. The cohort were split into two groups namely, injected (n=4) and non-injected (control, n=3) groups. The injected group were intravenously injected with nanoemulsion (200 μL of 155-229 mg/kg) 24 h before the animals were sacrificed by CO₂ asphyxiation followed by cervical dislocation to confirm death. At 24 h post injection, major organs of the injected and non-injected cohort (tumor, liver, spleen and muscle) were harvested and imaged with the MSOT for ex vivo bio distribution assessment. Organs were imaged in groups (injected and non-injected together) and in one measurement. Ultrasound colorless gel (approximately 0.5-1 mm think layer) was applied onto the clear plastic membrane for improved acoustic coupling. The organs were aligned horizontally onto the clear plastic membrane, injected (left) and non-injected (right) group were aligned side by side, having enough spacing in between the organ. The organs were immersed in a 34° C. water bath. The transducer was scanned from left to right direction of the samples. OA intensities were obtained from 680-900 nm, in 10 nm wavelength step and 1 mm step size using 25 mm field of view (FOV) taking 10 averages per frame.

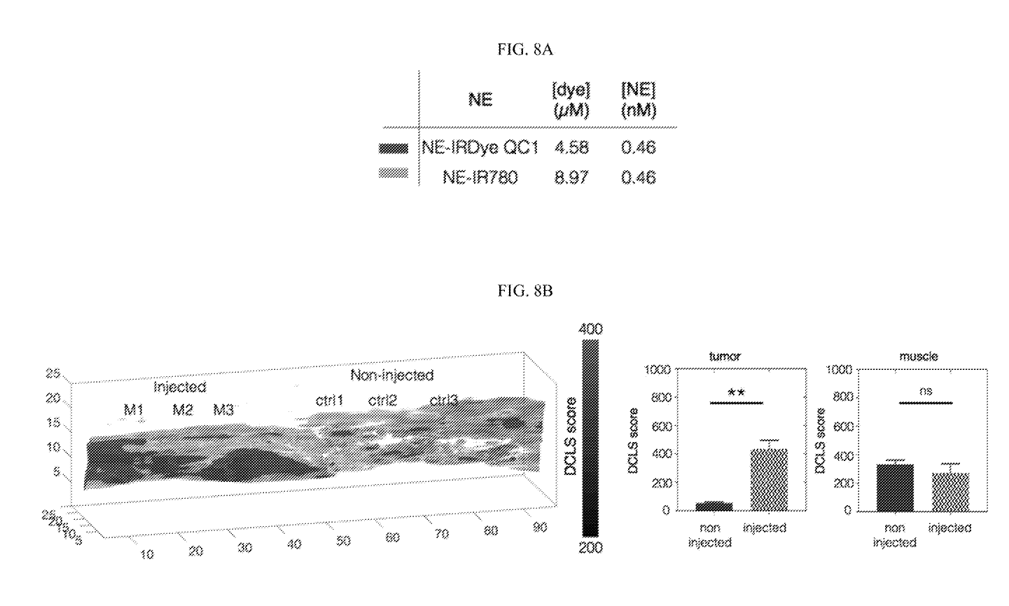

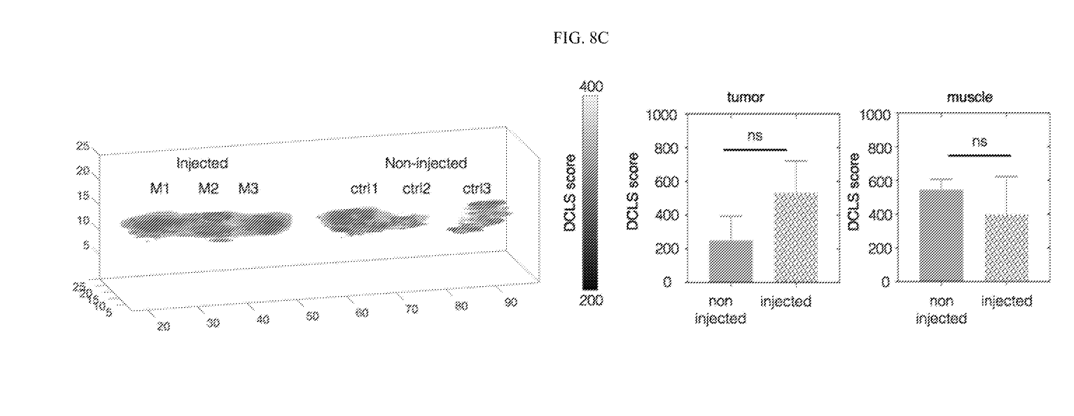

For mice injected with nanoemulsions (NE) having IRDye QC1 (See FIG. 8A), MSOT imaging of excised tumors revealed higher intensities and optoacoustic direct least squares (OA-DCLS) scores compared to the control group (FIG. 8B). The mean ratio of OA-DCLS scores ratio between injected to non-injected mice was 7.6±2.2. For nanoemulsions having IR780, the average signal was higher for the injected group. However, the injected to non-injected signal ratio was not statistically significant (p=0.11, FIG. 8C). When looking at the intensities and scores derived from muscle tissue, there were no statistically significant differences between the injected and non-injected groups for either of NE-IRDye QC1 (p=0.2) and NE-IR780 (p=0.34), and the OA-DCLS score ratios were 0.82±0.12 and 1.34±0.09 respectively.

In the case of excised tumors, imaged ex vivo, both nanoemulsions served to increase the OA signal vs. tumors from non-injected animals, as shown in FIG. 8A-8C. NE-IRDye QC1 (FIG. 8B) gave statistically significant difference (p<0.005), whereas for NE-IR780 (FIG. 8C) the increase in signal was not statistically significant (p=0.11).

In Vivo Optoacoustic Imaging

For in vivo MSOT experiments, a total of four female homozygous Hsd:athymic mice Nude-Foxn1^{nu} (6-8 weeks old) were used for the experiment. The mice were placed into the animal holder in supine position, gently fixed into position using clear straps and fitted with a breathing mask delivering a constant flow of 1.5-2% isoflurane anesthesia. Ultrasound colorless gel (approximately 1-2 mm thick layer) was applied onto the mouse around the region of interest in order to improve acoustic coupling. The animal holder was closed, wrapping the clear plastic membrane around the mouse, and inserted into the imaging chamber. The animal was aligned until the illumination ring coincides with the detection plane, centering the animal in the transducer array. To acquire a baseline scan, mice were scanned initially on the day prior to injection of contrast agent. Then, the animals were administered 155-229 mg/kg amount of concentration 0.47 nM nanoemulsion formulation and scanned again 24 h post injection. The scan parameters used for imaging animals were as reported above for phantoms. To minimize the scan duration for the animals, the spectral resolution was limited to 20 nm, and the longitudinal spatial resolution to 1 mm. The abdomen and hind limbs of the animals were scanned. Typical scan duration was 13 minutes. The animals were euthanized by carbon dioxide (CO₂) asphyxiation followed by cervical dislocation.

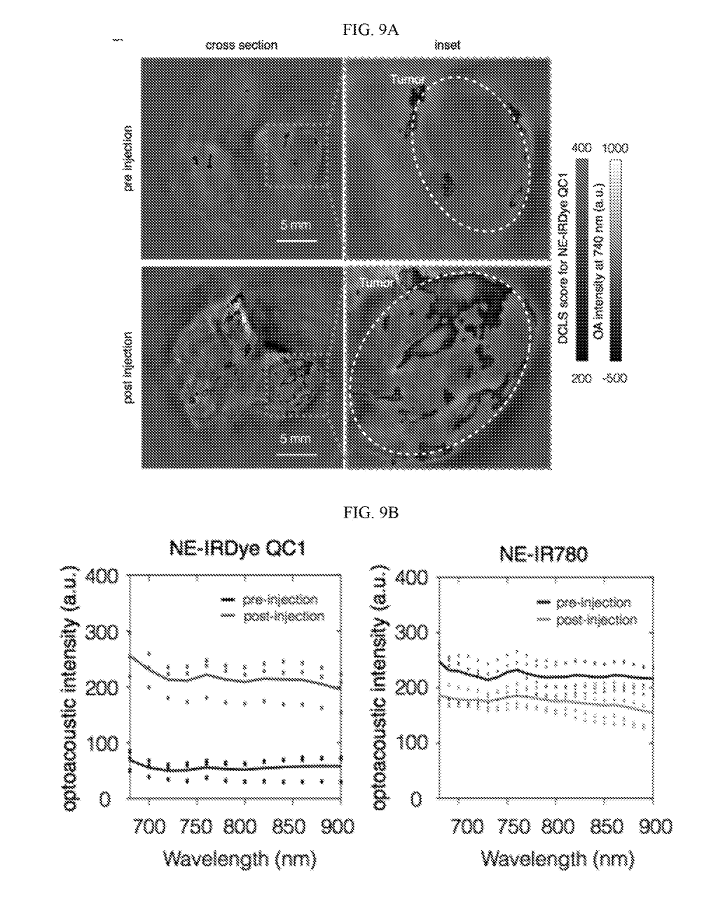

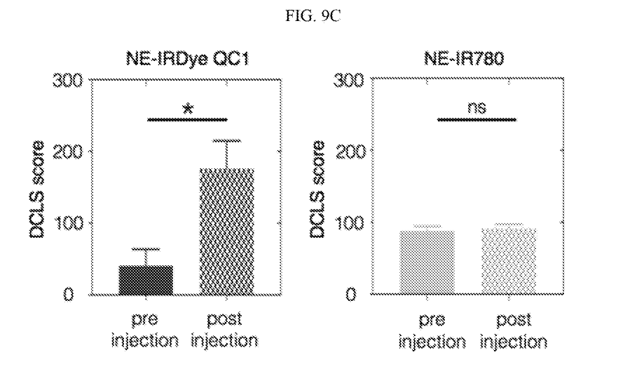

FIG. 9A shows a z-slice with a tumor, with the DCLS scores overlaid on top of the optoacoustic intensities (740 nm). The tumor from the same animal is shown before injection (top) and 24 h after (bottom). For NE-IRDye QC1, the overall OA intensity, shown in FIG. 9B, was found to be higher in the 24 h post injection mice as compared to their pre-injected counterparts. The corresponding OA-DCLS scores, shown in FIG. 9C, were found to increase four-fold post injection (p≤0.005). Conversely, the acquired data for the NE-IR780 exhibited no statistically significant differences in the MSOT intensity and DCLS score before and after injection (p=0.11).

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such
A. A composition comprising
   at least one contrast agent covalently linked to a targeting agent;
   wherein:
      the at least one contrast agent comprises a dark quencher;
      the targeting agent comprises an antibody, an antigen, an antigen-targeting ligand, a receptor ligand, or an adhesion peptide; and
      the at least one contrast agent and targeting agent are covalently linked by a linker of formula (I),

X¹-L¹-X² (I),
wherein:
   X¹ is an amino, amide, S, or O of the at least one contrast agent;
   L¹ is C₁-C₁₂ alkylene, C₂-C₁₂ alkenylene, C₅-C₁₂ cycloalkylene, or C₆-C₁₂ arylene; and
   X² is an amino, amide, S, or O of the targeting agent.
B. The composition of Paragraph A, wherein the dark quencher comprises a black hole quencher, dimethylaminoazobenzenesulfonic acid (Dabcyl), IRDye QC-1, DYQ-4, DYQ-700, Qx1 quencher, Iowa black FQ, Iowa black RQ, Atto 540Q, Atto 575Q, Atto 580Q, Atto 612Q, BHQ-0, BHQ-1, BHQ-2, BHQ-3, BHQ-10, BBQ-650, Ellipse, DYQ1, DYQ2, DYQ3, DYQ4, DYQ-425, DYQ505, DYQ700, DYQ660, or DYQ661.
C. The composition of Paragraph A or Paragraph B, wherein the at least one contrast agent further comprises a near infrared fluorescent dye.
D. The composition of any one of Paragraphs A-C, wherein the fluorescent dye is selected from the group consisting of cyanine 7 (Cy7) dye, cyanine 7.5 (Cy7.5) dye, IR780 dye, IR140 dye, Atto740 dye, DY-700 dye, DiR dye, and indocyanine green (ICG).
E. The composition of any one of Paragraphs A-D, wherein the targeting agent is selected from the group consisting of an antibody, an antigen-targeting ligand, or an adhesion peptide.
F. The composition of any one of Paragraphs A-E, wherein the targeting agent is a RGD peptide, a cRGD peptide, or a heparin-binding peptide.
G. An nanoemulsion composition comprising a plurality of lipid micelles in an aqueous phase, wherein
   each lipid micelle comprises
      an interior and an exterior;
      an outer amphiphilic component in contact with the aqueous phase on the exterior and with an oil phase on the interior; and
      at least one contrast agent within the interior; and
   the plurality of lipid micelles exhibit an intensity-weighted average particle diameter of about 100 nm to about 500 nm as determined by dynamic light scattering.
H. The nanoemulsion of Paragraph G, wherein the outer amphiphilic component comprises a fatty acid, a phospholipid, pegylated phospholipids, cholesterol, 1,2-diestearoyl-sn-glycero-3-phosphocholine, pegylated 1,2-distearoyl-sn-glycero-3-phosphoethanolamine, 1,2-dipalmitoyl-sn-glycero-3-phosphocholine, 1,2-distearoyl-sn-glycero-3-phosphoethanolamine-N-[amino(polyethylene glycol)-2000](ammonium salt), 1,2-distearoyl-sn-glycero-3-phosphoethanolamine-N-[folate(polyethylene glycol)-2000] (ammonium salt), or a combination of any two or more thereof.
I. The nanoemulsion of Paragraph G or Paragraph H, wherein the at least one contrast agent is at least one member selected from the group consisting of a dark quencher and a fluorescent dye.
J. The nanoemulsion of any one of Paragraphs G-I, wherein the interior of the lipid micelle further comprises a composition according to any one of claims 1-6.
K. The nanoemulsion of any one of Paragraphs G-J, wherein the aqueous phase comprises a phosphate buffer.
L. The nanoemulsion of any one of Paragraphs G-K, wherein the oil phase comprises at least one of medium chain triglycerides, peanut oil, sesame oil, soybean oil, cottonseed oil, corn oil, olive oil, ethyl oleate, isopropyl myristate, fatty acid glycerides, and acetylated fatty acid glycerides.
M. The nanoemulsion of any one of Paragraphs G-L, wherein the plurality of lipid micelles exhibit an intensity-weighted average particle diameter of about 110 nm to about 150 nm.
N. A method for obtaining an optoacoustic image, the method comprising:
   contacting a target with a composition according to any one of Paragraphs A-F or a nanoemulsion of any one of Paragraphs G-M;
   contacting the target with light, wherein the light has a wavelength in the visible to near infrared spectrum; and
   detecting ultrasound waves from the target.
O. The method of Paragraph N, wherein the target is a tumor.
P. The method of Paragraph 0, wherein the tumor comprises endothelial cells, epithelial cells, glioblastoma cells, breast cancer cells, pancreatic cancer cells, bladder cancer cells, and combinations of any two or more thereof.
Q. The method of Paragraph O or Paragraph P, wherein cells of the tumor express αᵥβ₃-integrin.
R. The method of any one of Paragraphs N-Q, wherein the light has a wavelength from about 500 nm to about 900 nm.
S. The method of any one of Paragraphs N-R, wherein the detecting comprises raster-scan optoacoustic mesoscopy.
T. A method for obtaining an optoacoustic image in a subject, the method comprising:
   administering to the subject an effective amount of a composition according to any one of Paragraphs A-F or an effective amount of a nanoemulsion of any one of Paragraphs G-M;
   contacting a target area of the subject with light, wherein the light has a wavelength in the visible to near infrared spectrum; and
   detecting ultrasound waves from the target area of the subject.
U. The method of Paragraph T, wherein the subject is a human suffering from or suspected of having cancer.
V. The method of Paragraph T or Paragraph U, wherein the target comprises a tumor.
W. The method of Paragraph V, wherein the tumor comprises endothelial cells, epithelial cells, glioblastoma cells, breast cancer cells, pancreatic cancer cells, bladder cancer cells, and combinations of any two or more thereof.
X. The method of Paragraph V or Paragraph W, wherein cells of the tumor express αᵥβ₃-integrin.
Y. The method of any one of Paragraphs T-X, wherein the light has a wavelength from about 500 nm to about 900 nm.
Z. The method of any one of Paragraphs T-Y, wherein the detecting comprises raster-scan optoacoustic mesoscopy.

Other embodiments are set forth in the following claims.

1. A composition comprising
at least one contrast agent covalently linked to a targeting agent;
wherein:
the at least one contrast agent comprises a dark quencher;
the targeting agent comprises an antibody, an antigen, an antigen-targeting ligand, a receptor ligand, or an adhesion peptide; and
the at least one contrast agent and targeting agent are covalently linked by a linker of formula (I),
X¹-L¹-X² (I),
wherein:
X¹ is an amino, amide, S, or O of the at least one contrast agent;
L¹ is C₁-C₁₂ alkylene, C₂-C₁₂ alkenylene, C₅-C₁₂ cycloalkylene, or C₆-C₁₂ arylene; and
X² is an amino, amide, S, or O of the targeting agent.

2. The composition of claim 1, wherein the dark quencher comprises a black hole quencher, dimethylaminoazobenzenesulfonic acid (Dabcyl), IRDye QC-1, DYQ-4, DYQ-700, Qx1 quencher, Iowa black FQ, Iowa black RQ, Atto 540Q, Atto 575Q, Atto 580Q, Atto 612Q, BHQ-0, BHQ-1, BHQ-2, BHQ-3, BHQ-10, BBQ-650, Ellipse, DYQ1, DYQ2, DYQ3, DYQ4, DYQ-425, DYQ505, DYQ700, DYQ660, or DYQ661.

3. The composition of claim 1, wherein the at least one contrast agent further comprises a near infrared fluorescent dye.

4. The composition of claim 1, wherein the fluorescent dye is selected from the group consisting of cyanine 7 (Cy7) dye, cyanine 7.5 (Cy7.5) dye, IR780 dye, IR140 dye, Atto740 dye, DY-700 dye, DiR dye, and indocyanine green (ICG).

5. The composition of claim 1, wherein the targeting agent is selected from the group consisting of an antibody, an antigen-targeting ligand, or an adhesion peptide.

6. The composition of claim 1, wherein the targeting agent is a RGD peptide, a cRGD peptide, or a heparin-binding peptide.

7. A nanoemulsion composition comprising a plurality of lipid micelles in an aqueous phase, wherein
each lipid micelle comprises
an interior and an exterior;
an outer amphiphilic component in contact with the aqueous phase on the exterior and with an oil phase on the interior; and
at least one contrast agent within the interior; and
the plurality of lipid micelles exhibit an intensity-weighted average particle diameter of about 100 nm to about 500 nm as determined by dynamic light scattering.

8. The nanoemulsion of claim 7, wherein the outer amphiphilic component comprises a fatty acid, a phospholipid, pegylated phospholipids, cholesterol, 1,2-distearoyl-sn-glycero-3-phosphocholine, pegylated 1,2-distearoyl-sn-glycero-3-phosphoethanolamine, 1,2-dipalmitoyl-sn-glycero-3-phosphocholine, 1,2-distearoyl-sn-glycero-3-phosphoethanolamine-N-[amino(polyethylene glycol)-2000] (ammonium salt), 1,2-distearoyl-sn-glycero-3-phosphoethanolamine-N-[folate(polyethylene glycol)-2000] (ammonium salt), or a combination of any two or more thereof.

9. The nanoemulsion of claim 7, wherein the at least one contrast agent is at least one member selected from the group consisting of a dark quencher and a fluorescent dye.

10. The nanoemulsion of claim 7, wherein the interior of the lipid micelle further comprises a composition according to claim 1.

11. The nanoemulsion of claim 7, wherein the aqueous phase comprises a phosphate buffer.

12. The nanoemulsion of claim 7, wherein the oil phase comprises at least one of medium chain triglycerides, peanut oil, sesame oil, soybean oil, cottonseed oil, corn oil, olive oil, ethyl oleate, isopropyl myristate, fatty acid glycerides, and acetylated fatty acid glycerides.

13. The nanoemulsion of claim 7, wherein the plurality of lipid micelles exhibit an intensity-weighted average particle diameter of about 110 nm to about 150 nm.

14. A method for obtaining an optoacoustic image, the method comprising:
contacting a target with a composition of claim 1;
contacting the target with light, wherein the light has a wavelength in the visible to near infrared spectrum; and
detecting ultrasound waves from the target.

15. The method of claim 14, wherein the target is a tumor.

16. The method of claim 15, wherein the tumor comprises endothelial cells, epithelial cells, glioblastoma cells, breast cancer cells, pancreatic cancer cells, bladder cancer cells, and combinations of any two or more thereof.

17. The method of claim 16, wherein cells of the tumor express αᵥβ₃-integrin.

18. The method of claim 14, wherein the light has a wavelength from about 500 nm to about 900 nm.

19. The method of claim 14, wherein the detecting comprises raster-scan optoacoustic mesoscopy.

20. A method for obtaining an optoacoustic image in a subject, the method comprising:
administering to the subject an effective amount of a composition of claim 1;
contacting a target area of the subject with light, wherein the light has a wavelength in the visible to near infrared spectrum; and
detecting ultrasound waves from the target area of the subject.

21.-26. (canceled)
